(19) United States
(12) Patent Application Publication
(10) Pub. No.: US 2004/0180383 A1
(43) Pub. Date: Sep. 16, 2004

(54) Methods for predicting the predisposition of an individual to total joint replacement failure

(76) Inventor: Erin Lynn Boynton, Toronto (CA)

Correspondence Address: Adrian M. Kaplan, Dimock Stratton Clarizio LLP, Suite 3202, Box 102, 20 Queen Street West, Toronto, ON, M5H 3R3, CA

(21) Appl. No.: 10/718,601

(22) Filed: Nov. 24, 2003

Related U.S. Application Data
(60) Provisional application No. 60/428,718, filed on Nov. 25, 2002.

(51) Int. Cl. G01N 33/53; G01N 33/567

(52) U.S. Cl. 435/7.2; 435/7.1

(57) ABSTRACT

Diagnostic methods are described for using the concentration of a cytokine and/or enzyme present in serum or other biological fluids in order to predict the predisposition of an individual to total joint replacement failure. Also described are diagnostic kits that are particularly suitable for use in such diagnosis.

### FIELD OF THE INVENTION

The present invention is in the field of diagnostics, and more specifically relates to a method and kit useful for predicting the predisposition of an individual to total joint replacement failure.

### BACKGROUND

Aseptic loosening remains the number one cause of total joint replacement failure. Submicron particles of the joint replacement material, such as polyethylene (PE), are generated by the normal wear of the implant surface. The interaction of PE particles with monocyte-macrophages at the bone implant interface results in chronic inflammation which ultimately destroys peri-implant bone leading to mechanical failure of the implant. As total joint replacement remains the most successful method of restoring quality of life for patients suffering from end-stage arthritis, preventing failure of the implant is important. Total joint replacements have remarkably improved the physical function, social interaction, and over-all health of patients. The quality of patients' lives improves markedly at the first three months post surgery, and is maintained for at least the next twenty-one months(¹). The long-term clinical application of implants is not yet satisfactory, as the loosening rates increase with time. By 15 years, 50% of implants have been reported to be radiographically loose in the absence of infection(²). Loosened prostheses may be tolerated for some time and function well, but the development of pain necessitates revision surgeries often within the next five to ten years. At the University of California in Los Angeles, revision procedures comprise up to 25% of all arthroplasty cases(³). In general, the rate of revision for sepsis or instability represents less than 1% of the annual cases. When assessing the need for revision due to aseptic loosening, it has been found that the rate is near 9% of all joint replacements. This implies that between 27,000 to 36,000 revision procedures, due to aseptic loosening, were required in 1993 in North America alone(⁴).

Since the inception of total joint replacement, the generation of PE wear particles has been recognized, and the problem has been addressed with some success. This has been achieved by working with different materials (e.g. porous metal in U.S. Pat. No. 5,973,222 and ceramic-metal composite in U.S. Pat. No. 5,711,763), altering the process of the implant material (e.g. ultra-high molecular weight PE in U.S. Pat. No. 6,242,507 and U.S. Pat. No. 6.048,480) and changing manufacturing protocols (e.g. solid phase deformation to orient polymer chains of PE in U.S. Pat. No. 6,146,426). Efforts have also been placed at minimizing a host's interaction with wear particles once they are generated. Membranous enclosures have been proposed for use with articulate hip joint replacements which isolate or encapsulate the joint (U.S. Pat. No. 3,683,421 and U.S. Pat. No. 3,739,403). A barrier comprising a biocompatible membrane that permits full motion of the replacement joint while preventing or impeding tissue and debris from migrating to and from bone implant interfaces has also been proposed (U.S. Pat. No. 6,132,470). Regardless of the techniques used to minimize wear particles or their interaction with the host, they cannot be completely eliminated and therefore understanding the mechanisms leading to the inflammatory response to implant particles remain crucial. Despite the fact that all implants generate particles, not all patients exhibit a serious inflammatory reaction to the particles. A number of particle factors such as the size, number, surface chemistry and volume will influence the inflammatory response. In addition, it is apparent that patient factors play a significant role in predisposing an individual to an inflammatory reaction to particulate, thus, aseptic loosening. Therefore, the ability to predict which patients are genetically predisposed to joint replacement failure secondary to the inflammatory response to implant wear debris would be advantageous.

### SUMMARY OF THE INVENTION

In the present invention, patient candidates for total joint replacement are first screened to detect any predisposition to total joint replacement failure. This is achieved using an assay that reveals changes in the levels at which pro-inflammatory mediators are produced by the patient's own peripheral blood monocyte/macrophages (PBMs), when those PBMs are challenged by particles of the joint replacement material chosen for use in the patient. Using this assay, patient candidates predisposed to total joint replacement are identified as those producing proinflammatory mediators at a level that is at least twice the level produced by patient candidates having no such predisposition. Alternatively, patients having such a predisposition can be identified as those producing inflammatory mediators at a level that is statistically no different from the level produced by patients experiencing total joint replacement failure. In addition, when the assay is performed using an increasing volume of particulate material relative to a fixed number of PBMs, patients having such a predisposition are revealed by a distinctively, bell-shaped dose reponse curve.

Thus, in one of its aspects, the present invention provides a method for assessing a patient for predisposition to total joint replacement failure, comprising the steps of:

a) assaying a patient sample containing monocytes/macrophages by measuring the level at which at least one pro-inflammatory marker is produced in response to incubation with a fixed or varied volume of a particulate form of the joint replacement material; and

b) comparing that measured level with either (i) a first reference level established for a population of primary patients or (ii) a second reference level established for a population of revision patients;

c) wherein a patient is identified as having a predisposition to total joint replacement if (a) the measured level is at least twice the first reference level, or (b) if the measured level is statistically no different from the second reference level, or (c) if the the level when measured at said varied volume is characterized by a bell-shaped dose response curve.

As used herein, a population of primary patients refers to a group (n=5 or more) of patients about to undergo total joint replacement. A population of revision patients refers to a group (n=5 or more) of patients experienced with total joint replacement failure secondary to aseptic loosening. Reference levels are the average levels at which these patient populations produce proinflammatory mediators, as measured by the assay herein described.

The pro-inflammatory markers that can usefully be measured in accordance with the present invention include the cytokines, and preferably interleukin-6 (IL-6), interleukin-1B (IL-1B) and tumor necrosis factor alpha (TNFa), as well as enzymes, including tartrate resistant acid phosphatase (TRAP).

Desirably, the assay used in the present method is adapted to measure the level of at least two or more proinflammatory mediators produced by the patient sample, and the present method entails correlation of those levels to the reference population, to identify patients predisposed to total joint replacement failure as those patients having a profile of proinflammatory mediator production that is statistically different from primary patients or statistically no different from revision patients.

The proinflammatory mediators are suitably detected as secreted products, using established ELISA methods or their equivalents. Alternatively, the proinflammatory mediators can be detected as mRNA transcripts, also using methods established for amplification of the targeted gene sequences, such as PCR in any of its various and applicable forms.

The particulate form of the joint replacement material used in the assay of the present method suitably simulates the particulate forms commonly detected in actual recipients of the joint replacement. Because of its popularity in today's joint replacements, the particles are desirably of replacement-grade polyethylene. It will be appreciated, however, that the assay can be performed with any other material used to fabricate the joint replacement to be introduced to a given patient.

The present invention further provides kits that are useful to perform the present method, which comprises some or all of the reagents suitable for conducting the assay, together with instructions for drawing the correlations that are diagnostic of total joint replacement failure. Such kits may, for instance, comprise one or more particulate formulations such as glass coverslips having the particulates adhered thereto, each containing a desired volume of particulates, for incubation with PBMs extracted from the patient.

### BRIEF DESCRIPTION OF THE DRAWINGS

FIG. 1 is a plot showing that absolute cytokine levels from the 20% of the patients receiving a primary implant who showed an increase in cytokine secretion after PE challenge, were still lower than the lowest absolute cytokine levels in the failed implant population;

FIG. 2 is a graph showing particle dose response following 18 hr incubation of monocytes with PE. This graph represents the IL-1β data, this pattern of cytokine response was also observed in TNF-α and IL-6 secretion;

FIG. 3 is a plot showing non implant cytokine response;

FIG. 4 is a plot showing failed implant cytokine response;

FIG. 5 is a block diagram showing cytokine response in failed implants versus non-implants;

FIG. 6(a) is a plot showing cytokine correlations for TNF-α versus IL-6 in non-implant patients.

FIG. 6(b) is a plot showing cytokine correlations for IL-1β versus IL-6 in non-implant patients.

FIG. 7(a) is a plot showing cytokine correlations for TNF-α versus IL-6 in failed-implant patients.

FIG. 7(b) is a plot showing cytokine correlations for IL-1β versus TNF-α in failed-implant patients.

### DETAILED DESCRIPTION OF THE INVENTION

Thus, in embodiments, the present invention concerns the use of cytokine and/or enzyme levels to diagnose the predisposition of an individual to total joint replacement failure. The invention is additionally directed to diagnostic kits suitable for use in this method.

The invention provides a method for determining the predisposition of an individual to total joint replacement failure. In one aspect, the method involves exposing monocyte-macrophages to particulate wear debris at varying particle volumes (ranging from 100:1 to 1:1 particles/cell) and measuring the cytokine and/or enzyme levels that are secreted by the cells at each concentration. The cytokine and/or enzyme levels of an individual that is predisposed to total joint replacement failure will be significantly higher than those levels of a healthy individual, i.e., a primary patient having no such predisposition. By primary patient, reference is made to a patient that has not previously received total joint replacement but who, by their symptoms, is a candidate for such replacement. In particular, cytokine and/or enzyme levels of an individual that is predisposed to total joint replacement failure will be at least twice as high as those levels of a healthy individual, preferably three times as high.

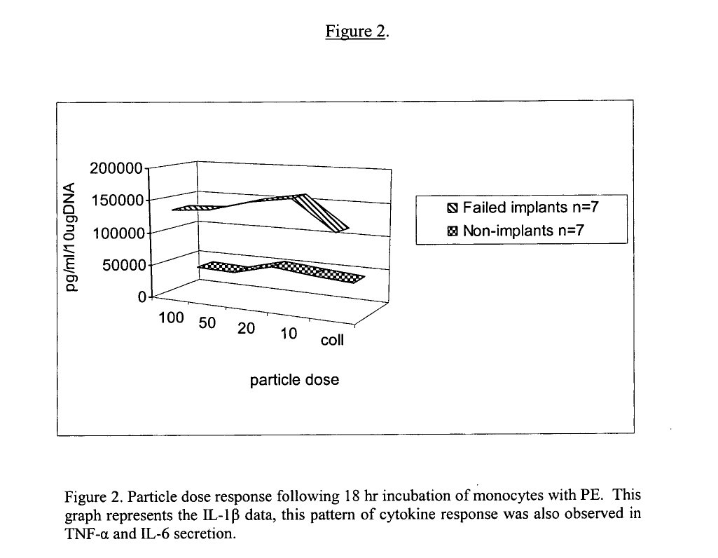

More specifically, the cytokine and/or enzyme levels of an individual that is predisposed to total joint replacement failure increase as the volume of particles increase until the cells are saturated with particles and then, the cytokine and/or enzyme levels decrease (e.g. the dose-response curve is bell shaped). In contrast, the cytokine and/or enzyme levels of a healthy individual remain relatively constant over the range of particle volumes tested (e.g. the dose-response curve is flat)[for example, see FIG. 2].

The invention particularly pertains to the embodiments wherein the particulate wear debris exploited in the assay is comprised of a polymer, metal or ceramic. Such particulate wear debris can include, but is not limited to, polyethylene, polymethylmethacrylate, titanium and its alloys, cobalt and its alloys, aluminum oxide and zirconium oxide.

The invention is directed in certain embodiments, to the use of an immunoassay to determine cytokine and/or enzyme concentration at each particle volume. Interleukin-1 (IL-1), interleukin-6 (IL-6) and tumor necrosis factor-alpha (TNF-α) are the preferred cytokines to measure, however, it is appreciated that any protein that is involved in mediating an inflammatory response can be used. Tartrate-resistant acid phosphatase (TRAP) is the preferred enzyme to measure.

I. The Correlation Between Cytokine Concentration and Predisposition to Total Joint Replacement Failure

Genetic Predisposition to Particle Induced Inflammation

The inflammatory response to a stimulus such as wear particulate is designed to allow for tissue repair and wound healing at the bone implant interface without damage to the host. Incorporation of an implant would thus depend upon the balance between pro- and anti-inflammatory cytokines which is regulated at several levels in both a paracrine and autocrine fashion. With loss of the normal macrophage autoregulation a prolonged inflammatory response ensues and in the case of total joint replacements, the infiltration of inflammatory cells which result in peri-implant bone loss(⁵). As large numbers of monocyte/macrophages are found at inflammatory sites, dysregulation of the cytokines produced by these cells would have considerable consequences on the pathogenesis of inflammatory diseases. Macrophage derived cytokines have been implicated in disease progression(^{6;7}), and cytokine levels between individuals have been shown to have reproducible differences (^{8;9}). In addition, correlations have been found between polymorphisms in IL-1β, IL-6 and TNF-α genes and a range of inflammatory diseases. Therefore, defining the differences in cytokine synthesis and release following exposure to wear particulate and correlating these differences to implant failure will allow one to determine the “high cytokine responder” and the “low cytokine responder”.

Previous in vitro models to study the interaction of macrophages with particulate PE have utilized macrophage cell lines or animal macrophages. The advantage of this is the predictable behavior of the cells once exposed to PE particulate, it will not however, allow the investigation of the variability observed when human cells are tested. Therefore, a unique human monocyte-macrophage model was developed by Boynton et al. for these studies. Significant donor heterogeneity has been observed when human cells are studied, which may explain the variable levels in cytokine and enzyme levels observed after a PE particle challenge. Every individual has unique DNA, which can result in a range of responses to the same stimuli. In addition to the normal diversity found in DNA, genetic polymorphisms also exist. A polymorphism is a gene mutation that has reached a frequency of 2% in a population. Polymorphisms are often found in the regulatory regions of cytokine genes(⁵).

Cell Culture System to Study the Components Implicated in Osteolysis

In order to dissect the mechanism of inflammation associated with total joint replacement failure, a unique in vitro culture system to study the two key components implicated in osteolysis, the macrophage and polyethylene was developed by Boynton et al. (¹⁰). In this model, PE particles were first chemically characterized and endotoxin-tested. The chemical characterization of the particles showed features that were typical of polyethylene structure, and the endotoxin test revealed that the particles were free of endotoxin (¹⁰). Collagen type I was then applied to overcome the problems related to the hydrophobic and the low-density character of the polyethylene. A mixture of type I collagen with PE particles was solidified on glass coverslips, thus trapping PE particles onto the coverslips and facilitating adherent macrophage phagocytosis (¹⁰). Using this model, it was shown that mouse macrophages (IC-21 cell line) stimulated with HDPE particles (mean: 4.5 μm) released significantly higher levels of IL-1α, IL-1β, PGE₂, β-galactosidase, and hexosaminidase over collagen controls(¹⁰). Histological studies demonstrated the internalization of PE particulate and documented cell morphology and viability before and after PE phagocytosis (¹⁰).

Macrophage Cytokine Secretion Following Exposure to Wear Particles

For the present invention, the cell culture model developed by Boynton et al. was advanced to utilize human peripheral blood monocyte macrophages obtained from the patient undergoing screening for predisposition. The inflammatory cytokine and degradative enzyme profiles where then characterized using submicron particles of PE. During the characterization of the model it was noted that the absolute cytokine secretion levels varied quite markedly between individuals, with approximately 15% of the donors secreting 3-4 times higher cytokine levels compared to the remainder of the population. This indicated a likely genetic predisposition to an over exuberant inflammatory response to PE wear particles which could correlate with implant failure.

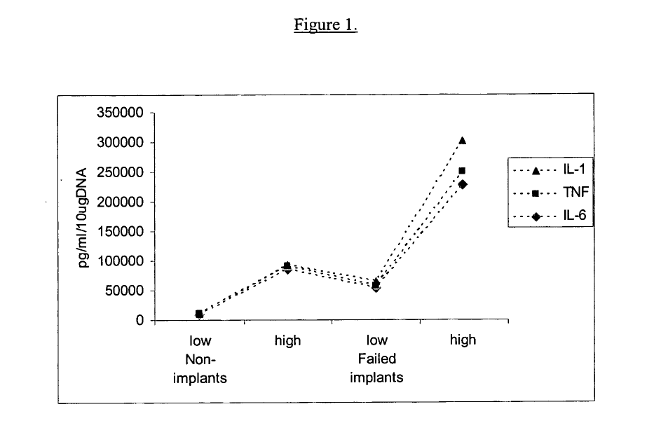

A comparison of macrophage cytokine secretion following a PE challenge was made between osteoarthritic patients with aseptic loosening of a total joint to healthy patients scheduled to receive a primary implant. A dramatic elevation in absolute cytokine secretion was observed in the patients with frank failure of a total joint replacement. All of the patients with osteolysis, compared to only 20% of the patients receiving primary implants, showed an increase in cytokine secretion as the volume of particulate increased. In the patients with osteolysis, the cytokine levels increased as the volume of PE increase (100 to 1, 50 to 1, 20 to 1, 10 to 1, and 1 to 1 particles/cell) until the cells were saturated with PE and then, cytokine levels dropped off (cell viability equal). In addition, it can be shown that the absolute cytokine levels from the 20% of the patients receiving a primary implant who showed an increase in cytokine secretion after PE challenge, were still lower than the lowest absolute cytokine levels in the failed implant population (FIG. 1). It is clear that there are significant differences in not only the absolute amount of cytokine secreted, but the pattern of cytokine secretion following a dose response challenge.

Therefore, the invention provides a method for determining the predisposition of an individual to total joint replacement failure. The method involves exposing monocyte-macrophages to particulate wear debris at varying particle volumes (ranging from 100:1 to 1:1 particles/cell) and measuring the cytokine (e.g. IL-1, IL-6, TNF-α) and/or enzyme (TRAP) levels that are secreted by the cells at each concentration. The cytokine and/or enzyme levels of an individual that is predisposed to total joint replacement failure will be significantly higher (at least twice, but preferably thrice) than those levels of a healthy individual. More specifically, the cytokine and/or enzyme levels of an individual that is predisposed to total joint replacement failure will increase as the volume of particles increase until the cells are saturated with particles and then, the cytokine and/or enzyme levels decrease (e.g. the dose-response curve is bell-shaped). In contrast, the cytokine and/or enzyme levels of a healthy individual remain relatively constant over the range of particle volumes tested (e.g. the dose-response curve is flat).

While PE is the biomaterial of choice in 80% of total joint replacements, other polymers, ceramics and metals are used. These biomaterials also generate wear particles which will elicit an inflammatory response that may ultimately lead to aseptic loosening and failure of the joint replacement. Therefore, it will be obvious to one skilled in the art that the method of determining the predisposition of an individual to total joint replacement failure can be extended from the use of particulate wear debris composed of PE, to other materials that are used for total joint replacements. These materials may include but are not limited to polymethylmethacrylate, cobalt and its alloys, stainless steel, titanium and its alloys, aluminum oxide and zirconium oxide.

The ability to determine pre-operatively if an individual is predisposed to total joint replacement failure will allow one to select a biomaterial with the least inflammatory potential for that specific individual, thus decreasing the risk of implant failure. Alternatively, if the patient is a “high responder” to all materials, and appears to be “at risk” for implant failure, then appropriate counseling could be conducted and treatment implemented to prevent chronic inflammation at the bone-implant interface.

II. Diagnostic Kits

The present invention includes articles of manufacture, such as “kits” that have been specially adapted to contain, reagents that facilitate the use of the above described methods.

Any of a variety of kits may be fashioned so as to facilitate the above described methods. In one embodiment, such kit may comprise a series of wells that are coated with a 1% collagen: particle solution at a volume of 1:1, 10:1, 20:1, 50:1 and 100:1. The amount of collagen solution which correlates to each dose of particle will be used alone as a negative control. The particles contained in the wells will consist entirely of polyethylene or will contain a combination of polyethylene with other materials in order to identify differences in patient sensitivity to various materials. Standard immunoassays can be used to measure cytokine and enzyme levels of the resulting supernatant solution that is collected up to 24 hours after culture.

The kits may also contain reagents, wash or substrate buffers, and the like, sufficient for multiple assays including standards and/or controls, as well as instructional brochures, etc.

Having now generally described the invention, the same will be more readily understood through reference to the following example which is provided by way of illustration, and is not intended to be limiting of the present invention, unless specified.

### EXAMPLE

Use of IL-6, IL-1 and TNF-α to Predict Total Joint Replacement Failure

PE Particle Solution and Coverslip Preparation

PE particles obtained from Howmedica Inc. were generated using a wear simulator and retrieved from the serum surrounding the apparatus. Using scanning electron microscopy (SEM) particles were characterized to be 1.86±0.87 μm in length and 0.75±0.25 μm in width and shapes included rods, spheres and fragments. PE particles were sterilized with 2.5 Mrad γ-irradiation and determined to be endotoxin free using an E-TOXATE detection kit (Sigma, St. Louis, Mo.). Particle-collagen solutions were prepared as previously described (8). Briefly, particles were suspended in 0.01% collagen type I solution (from calf skin, C-8919, Sigma, St. Louis, Mo.) at a concentration determined using the particle weight to volume ration for PE (1 μm³=1×10⁹ mg). Round, glass coverslips (15 mm diameter, Fisher Scientific, Whitby, Ont) were coated with 3.6 μl of the particle suspension; a serial dilution was performed on the particle solution, resulting in a range of particle volumes (100 to 1, 50 to 1, 20 to 1, and 10 to 1 particles/cell) adhered to the coverslip. Coverslips coated with the collagen solution alone were used as negative controls.

Patients

Two experimental groups were set up as follows: 1) patients undergoing revision surgery for a failed total hip replacement (n=7), and 2) patients undergoing primary total hip surgery for osteoarthritis of the hip (n=7). Primary indication for hip replacement surgery was osteoarthritis in all patients, both non-implant and failed implant population groups. Revision surgery was performed for aseptic loosening with ballooning osteolysis in the failed implant patients. Patients in both experimental groups were admitted to hospital the day of surgery and blood collected shortly after sedation in the operating room.

Cell Cultures

Human blood was collected in heparinized vacutainers (Becton Dickenson, Franklin Lakes, N.J.) from two different groups of volunteers (University of Toronto, ethical protocol #2015). The experimental groups were as follows: 1) patients undergoing revision surgery for a failed total hip replacement, and 2) patients undergoing primary total hip surgery for osteoarthritis of the hip. Lymphocytes were isolated using Ficoll-Paque (Pharmacia, Biotech) density gradient centrifugation and monocytes then isolated with adherence. Cells were cultured in 24-well tissue culture plates with RPMI 1640 media (R7509, Sigma) supplemented with 10% heat inactivated fetal bovine serum (Gibco BRL, Burlington, ON), 100 units/ml penicillin-streptomycin (Gibco BRL, Burlington, ON), 68 mM L-glutamine (Gibco BRL, Burlington, ON), and incubated at 37° C. in 5% CO² atmosphere. Two hours after cell seeding cell contents were aspirated and replaced with fresh media; this was defined at as time zero. Fourteen separate cultures were performed with seven patients in the failed implant group and seven patients in the non-implant group.

DNA Analysis

DNA contents of each well were quantified to normalize cytokine secretion to the amount of DNA (15;24). At time zero adherent cells were rinsed with PBS and incubated with lysis buffer (0.05% Triton X-100/10 mM EDTA/PBS). DNA content was determined with a fluorometric assay using Hoechst dye (Fisher H33258) as previously outlined by Labow et al. (14). Each DNA sample was assayed in duplicate.

Cytokine Analysis

Media conditioned by culturing with PBM was collected at 18 and 24 hours after incubation, centrifuged in a microcentrifuge at 2500 rpm for 5 minutes and stored as 200 μl aliquots at −70° C. until analysis by enzyme linked immunosorbant assay (ELISA). Commercially available ELISA kits were used to quantify cytokine levels: human IL-1β (KHC0012, Biosource International, Calif.), human IL-6/TNF-α Flexia™ (Biosource International). The absorbance was read at 450 nm using a VersaMax microplate reader (Molecular Devices) with SoftMax Pro software, version 3.1.1. All samples were analyzed in triplicate and the standards in duplicate. Cytokine values were then normalized with the DNA value obtained at time zero.

Statistical Analysis

Data was normally distributed and expressed as the mean±standard deviation. Analyze It© a program add-on for Microsoft Excel was used for the statistical analyses. A paired Student's t-test was applied to analyze the differences in cytokine secretion within experimental groups. One-way analysis of variance (ANOVA) was used to compare the means between experimental groups with Scheffe's post-hoc analysis to account for multiple comparisons. The relationships between cytokine levels were evaluated with the Pearson's Product Moment, a test to evaluate the linear relationship between variables. Statistical significance was determined at the 0.05 level (p<0.05).

Particle Dose Response

FIG. 2 shows a particle dose response following 18 hr incubation of monocytes with PE. This graph represents the IL-1β data, this pattern of cytokine response was also observed in TNF-α and IL-6 secretion. A dose response was seen for all three pro-inflammatory cytokines analyzed: IL-6, IL-1β and TNF-α. The cytokine response was roughly bell-shaped, and this pattern of distribution was demonstrated by all the study participants (see FIG. 2). The dose response curves in the failed implant patients demonstrated large differences in cytokine response to the various particle concentrations and collagen control. In contrast to this, the dose response curves seen in the non-implant population showed more subtle differences in cytokine secretion. As cytokine secretion appeared to peak between the 100 to 1 particle volume/cell number ratio and the 10 to 1 particle volume/cell number ratio, data from these ratios were selected to be the focus of this work.

Cytokine Release—Non-Implants

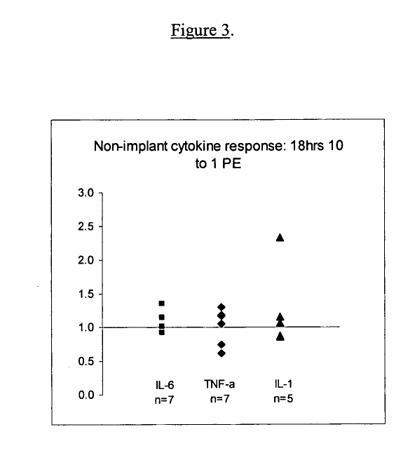

Cytokine data from the seven different donors was pooled and analyzed statistically as previously described. All cytokines were measured in triplicate for each individual. No significant increase in IL-6, TNF-α or IL-1β secretion was seen at either 18 or 24 hours for both the 100 to 1 and 10 to 1 particle volume/cell ratios in the pooled results when compared to the collagen control. When individual patient cytokine levels were evaluated at 18 hours, 3 of 7 had increased IL-6 and TNF-α secretion in response to the 10 to 1 PE ratio while one patient had elevated IL-1β (p<0.05) (FIG. 3). Although no significance was seen in the pooled results of the non-implant experimental group, the cytokine levels exhibited a large range in cytokine values demonstrating the heterogeneity present in the non-implant patient group.

With respect to the data shown in FIG. 3, cytokine levels for the 10 to 1 particle volume are depicted. Collagen control levels have been set to equal one (collagen=1), thus the points on the chart indicate the cytokine increase elicited by PE exposure. Cytokines release in response to PE was primarily equal to or less than that elicited by the collagen control. Table 1 sets out the data depicted in FIG. 3.

TABLE 1

| Patient | IL-6 | TNF-a | IL-1 |
|---|---|---|---|
| 1 | 2.4 | 2.2 | 2.3 |
| 2 | 0.9 | 1.2 | 1.2 |
| 3 | 1.4 | 0.6 | |
| 4 | 1.0 | 0.7 | 0.9 |
| 5 | 1.2 | 1.3 | 1.1 |
| 6 | 0.9 | 1.2 | 0.9 |
| 7 | 1.4 | 1.1 | |

Cytokine Release—Failed Implants

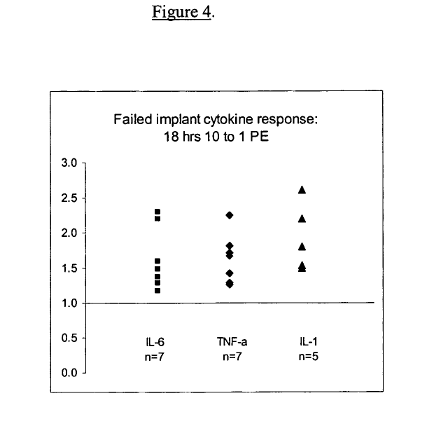

Cytokine data from the seven different donors was pooled and analyzed statistically as previously described. All cytokines were measured in triplicate for each individual. Analysis of the pooled IL-6 data showed that monocytes exposed to the 10:1 PE secreted a significantly higher amount of IL-6 compared to the collagen control (p<0.05). When individual patient cytokine levels were evaluated at 18 hours, 5 of 7 had increased IL-6 in response to 10 to 1 PE and 3 of 7 to the 100 to 1 PE ratio (FIG. 4). Statistical analysis of the TNF-α values demonstrated a significant increase of TNF-α in response to the 10 to 1 PE compared to the collagen control (p<0.05). When individual patient cytokine levels were evaluated at 18 hours, 5 of 7 had increased TNF-α in response to 10 to 1 PE ratio. Significant elevation of cytokine secretion was seen in response to both 100 to 1 PE and 10 to 1 PE (p<0.05). When individual patient cytokine levels were evaluated at 18 hours, 4 of 5 had increased IL-1β in response to 10 to 1 PE and 2 of 5 to the 100 to 1 PE ratio. Following the trend seen in the non-implant group the absolute level of cytokine secretion varied greatly between the individuals in the failed implant patient population.

With respect to FIG. 4, the specific data depicts cytokine levels for the 10 to 1 particle volume. Collagen control levels have been set to equal one (collagen=1), thus the points on the chart indicate the cytokine increase elicited by PE exposure. All patients in the failed implant groups had increased cytokine secretion in response to PE with respect to the collagen control. The data is summarized in Table 2.

TABLE 2

| Patient | IL-6 | TNF-a | IL-1 |
|---|---|---|---|
| 1 | 1.6 | 1.3 | 1.5 |
| 2 | 2.2 | 1.8 | 2.2 |
| 3 | 1.5 | 1.7 | 2.6 |
| 4 | 2.3 | 2.3 | 1.8 |
| 5 | 1.2 | 1.4 | 1.5 |
| 6 | 1.4 | 1.7 | |
| 7 | 1.3 | 1.3 | |

Cytokine Release—Comparison of Patient Populations

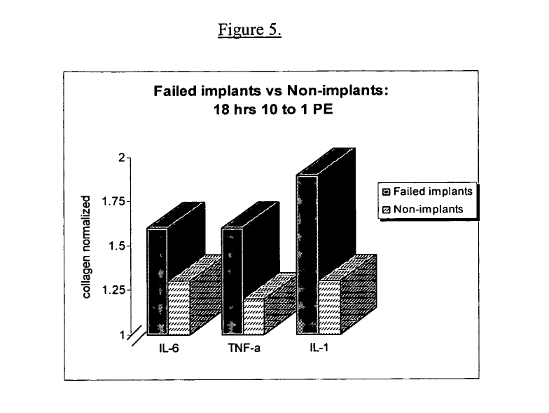

The cytokine levels for the three pro-inflammatory cytokines (IL-6, TNF-α, IL-1β), were compared between the two experimental groups, failed implants and non-implants, using a one-way ANOVA. The mean values of all three cytokines were significantly higher (p<0.05) in the failed implant group when compared to the non-implant patient population in all experimental conditions tested (100 to 1 and 10 to 1 PE ratios, collagen control) at both the 18 hr and 24 hr time points (FIG. 5). FIG. 5 depicts cytokine levels for the 10 to 1 particle volume. Collagen control levels have been set to equal one (collagen=1), thus the bars on the chart indicate the cytokine increase elicited by PE exposure.

Correlation Between Cytokine Levels

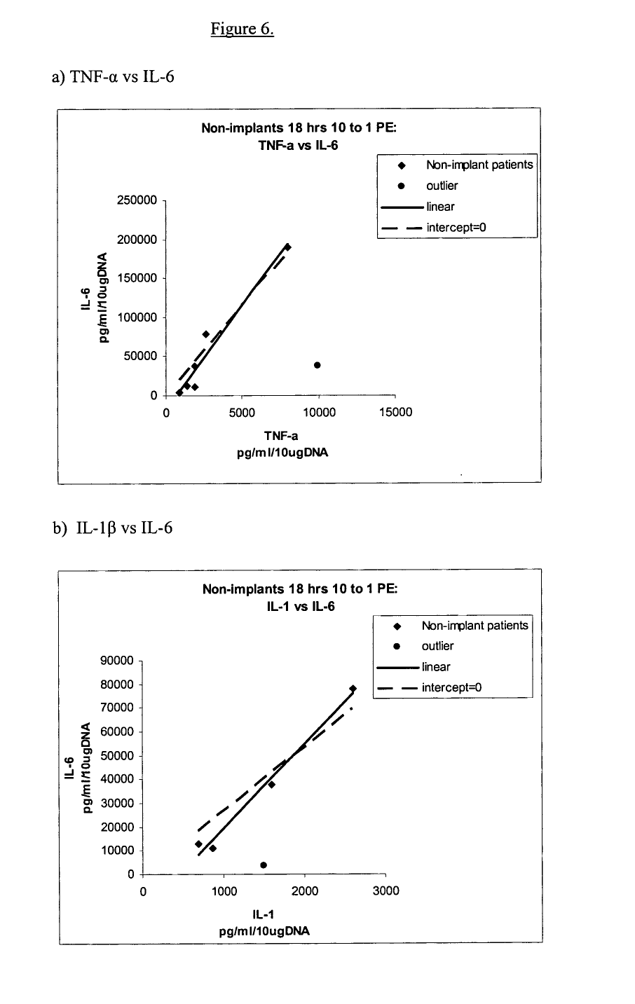

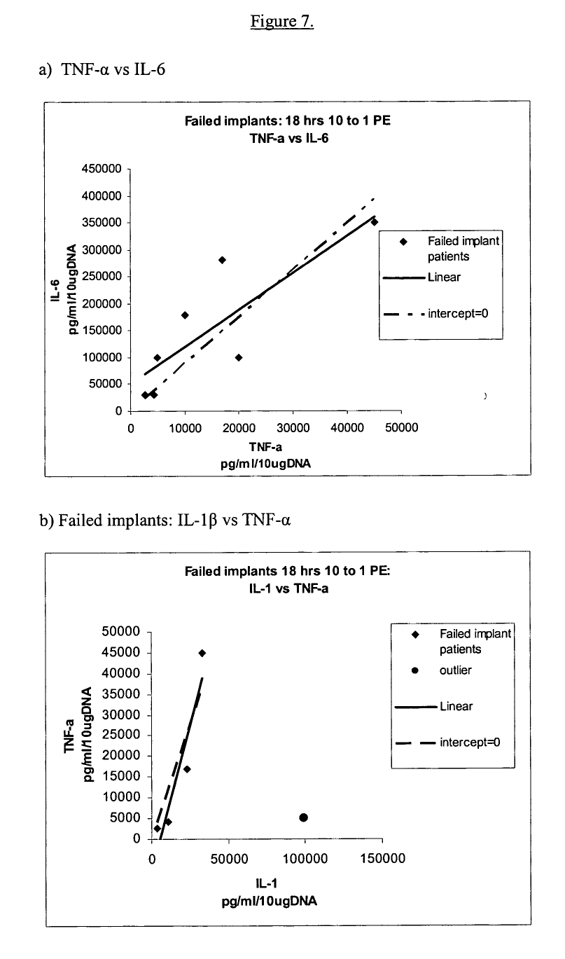

IL-6, IL-1β and TNF-α are pro-inflammatory cytokines and thus can be used to evaluate the inflammatory response to a material. Therefore, if one cytokine is elevated in response to in vitro PE exposure, it is possible other pro-inflammatory cytokines will follow a similar pattern. When the linear relationships between cytokine levels were tested with the Pearson's Product Moment, both the non-implant and failed implant patients demonstrated a significant relationship between their inflammatory cytokine levels: TNF-α versus IL-6 (p<0.05); TNF-α versus IL-1β (p<0.05) [FIGS. 6 and 7].

The following references cited herein are expressly incorporated herein by reference:

1. Boynton E L., Henry M., Morton J., and Waddell J P. The Inflammatory Response to Particulate Wear Debris in Total Hip Arthroplasty. Can J Surg. 38:507-515, 1995.

2. Mulroy R D, and Harris W H. The Effect of Improved Cementing Techniques on Component Loosening in Total Hip Replacement. An 11-year Radiographic Review. J Bone Joint Surg. 72B:757-760, 1990.

3. Amstutz H C., Ma S M., Jinnah R H., and Mai L. Revision of Aseptic Loose Total Hip Arthroplasties. Clin Orthop. 170:21-33, 1982.

4. Amstutz HC (ed). Hip Arthroplasty. New York, N.Y., Churchill Livingstone, 1991.

5. Cytokine. Engle Wood Cliffs, N.J., Prentice Hall, 1991.

6. Dinarello, C. A. and Wolff, S. M.: The role of interleukin-1 in disease. N. Engl. J. Med. 328:113, 1993.

7. Roodman, G. D. and et al: Interleukin 6. A potential autocrine/paracrine factor in Paget's disease of bone. J of Clin Invest. 89:52, 1992.

8. Matthews, J.B et al. Comparison of the response of primary human peripheral blood mononuclear phagocytes from different donors to challenge with model polyethylene particles of known size and dose. Biomaterials. 21:2044,2000.

9. Molvig, J. and et al: Endotoxin-stimulated human monocyte secretion of interleukin 1, tumour necrosis factor alpha, and prostaglandin E2 shows stable interindividual differences. Scand J Immunol. 27:716, 1988.

10. Voronov, I., Santerre, J. P., Hinek, A., Callahan, J. W., Sandhu, J., and Boynton, E. L.: Macrophage phagocytosis of polyethylene particulate in vitro. journal biomedical materials research. 39:40-51, 1998.

11. Xing S, Santerre J P, Labow R S, Boynton E L. Differential response to chemically altered polyethylene by activated mature human monocyte-derived macrophages. Biomaterials 2002; 23:3595-3602.

12. Voronov I, Santerre J P, Hinek A, Callahan J W, Sandhu J, Boynton E L. Macrophage phagocytosis of polyethylene particulate in vitro. J Biomed Mater Res 1998; 39(1):40-51.

13. Sabokbar A, Fujikawa Y, Neal S, Murray D W, Athanasou N A. Human arthroplasty derived macrophages differentiate into osteoclastic bone resorbing cells. Ann Rheum Dis 1997; 56(7):414-420.

14. Boynton EL, Waddell J E, Meek E, Labow R S, Edwards V, Santerre J P. The effect of polyethylene particle chemistry on human monocyte-macrophage function in vitro. J Biomed Mater Res 2000; 52(2):239-245.

Various embodiments of the present invention having been thus described in detail by way of example, it will be apparent to those skilled in the art that variations and modifications may be made without departing from the invention. The invention includes all such variations and modifications as fall within the scope of the appended claims.

We claim:

1. A method for assessing a patient for predisposition to total joint replacement failure, comprising the steps of:
a) assaying a patient sample containing monocytes/macrophages by measuring the level at which at least one pro-inflammatory marker is produced in response to incubation with a fixed or varied volume of a particulate form of the joint replacement material;
b) comparing that measured level with either (i) a first reference level established for a population of primary patients or (ii) a second reference level established for a population of revision patients; and
c) wherein a patient is identified as having a predisposition to total joint replacement if (a) the measured level is at least twice the first reference level, or (b) if the measured level is statistically no different from the second reference level, or (c) if the level when measured at said varied particulate volume is characterized by a bell-shaped dose response curve.

2. The method according to claim 1, wherein said levels using said varied volume of the particulate form of the joint replacement material to produce a dose response curve, and identifying patients predisposed to joint replacement failure as those patients for which a bell-shaped dose response curve is generated.

3. The method according to claim 1, wherein the particulate joint material is polyethylene.

4. The method according to claim 1, wherein the pro-inflammatory marker is selected from a cytokine and an enzyme.

5. The method according to claim 4, wherein the pro-inflammatory marker is selected from interleukin-6, interleukin-1B and tumour necrosis factor alpha.

6. The method according to claim 4, wherein the enzyme is TRAP.

7. The method according to claim 1, in which the levels of at least two pro-inflammatory markers are correlated, and compared with said reference levels to identify patients having said predisposition.

8. The method according to claim 1, wherein the level at which the pro-inflammatory marker is produced is measured by detecting the secreted form of that marker.

9. The method according to claim 1, wherein the level at which the pro-inflammatory marker is produced is measured by detecting transcripts of a gene encoding said marker.

10. A kit comprising one or more reagents for performing the assay defined according to any preceding claim, and instructions for assessing said predisposition based on the results of said assay.

11. A kit according to claim 10, comprising an assay substrate pre-loaded with particulate joint replacement material.

12. A kit according to claim 11, wherein said substrate comprises particulate joint replacement material pre-loaded with particulates in a range of different volumes.
